(19) United States
(12) Patent Application Publication
(10) Pub. No.: US 2011/0229509 A1
(43) Pub. Date: Sep. 22, 2011

(54) POLYPEPTIDES FOR INDUCING A PROTECTIVE IMMUNE RESPONSE AGAINST STAPHYLOCOCCUS AUREUS

(76) Inventors: Tessie B. McNeely, Gwynedd Valley, PA (US); Leslie D. Cope, Hatfield, PA (US); Mark A. Miller, Wyndmoor, PA (US); Loren D. Schultz, Harleysville, PA (US); Xinmin Wang, Schwenksville, PA (US)

(21) Appl. No.: 13/130,974

(22) PCT Filed: Nov. 18, 2009
(86) PCT No.: PCT/US2009/064925

§ 371 Date: May 24, 2011

Related U.S. Application Data
(60) Provisional application No. 61/200,273, filed on Nov. 26, 2008.

(51) Int. Cl. A61K 39/085; C07K 14/31; C07H 21/00; C12N 15/63; C12N 1/00; C12N 5/10; C12P 21/02; A61P 31/04; A61P 37/04

(52) U.S. Cl. 424/190.1; 530/350; 530/403; 536/23.7; 435/320.1; 435/243; 435/325; 435/69.3

(57) ABSTRACT

Disclosed are polypeptides comprising an amino acid sequence structurally related to SEQ ID NO 1 and uses of such polypeptides and compositions thereof. SEQ ID NO 1 is a full length S. aureus sequence. A derivative of SEQ ID NO 1 containing an amino terminal his-tag was found to produce a protective immune response against S. aureus.

### CROSS-REFERENCE TO RELATED APPLICATIONS

The present application claims the benefit of U.S. Provisional Application No. 61/200,273, filed Nov. 26, 2008, hereby incorporated by reference herein.

### BACKGROUND OF THE INVENTION

Staphylococcus aureus (“S. aureus”) is a bacterial pathogen responsible for a wide range of diseases and conditions. While S. aureus commonly colonizes in the nose and skin of healthy humans, often causing only minor infections (e.g., pimples, boils), it can also cause systemic infections. Examples of diseases and conditions caused by S. aureus include bacteremia, infective endocarditis, folliculitis, furuncle, carbuncle, impetigo, bullous impetigo, cellulitis, botryomyosis, toxic shock syndrome, scalded skin syndrome, central nervous system infections, infective and inflammatory eye disease, osteomyelitis and other infections of joints and bones, and respiratory tract infections. (The Staphylococci in Human Disease, Crossley and Archer (eds.), Churchill Livingstone Inc. 1997; Archer, 1998, Clin. Infect. Dis. 26:1179-1181.)

Normally, mucosal and epidermal barriers protect against S. aureus infections; however, both the interruption of these natural barriers as a result of injuries (e.g., burns, trauma or surgical procedures) and diseases that compromise the immune system (e.g., diabetes, end-stage renal disease, cancer) dramatically increase the risk of infection. Opportunistic S. aureus infections can become quite serious, often resulting in severe morbidity or mortality.

Methicillins, introduced in the 1960s, largely overcame the problem of penicillin resistance to S. aureus. However, methicillin resistance has emerged in S. aureus, along with resistance to many other antibiotics effective against this organism (e.g., aminoglycosides, tetracycline, chloramphenicol, macrolides and lincosamides). Methicillin-resistant S. aureus (MRSA) has become one of the most important nosocomial pathogens worldwide and poses serious infection control problems.

Immunological based strategies can be employed to control S. aureus infections and the spread of S. aureus. Immunological based strategies include passive and active immunization. Passive immunization employs immunoglobulins targeting S. aureus. Active immunization induces immune responses against S. aureus.

Potential S. aureus vaccines target S. aureus polysaccharides and polypeptides. Examples of polysaccharides that may be employed as possible vaccine components include S. aureus type 5 and type 8 capsular polysaccharides (Shinefield et al., 2002, N. Eng. J. Med. 346:491-496). Examples of polypeptides that may be employed as possible vaccine components include collagen adhesin, fibrinogen binding proteins, and clumping factor (Mama et al., 1994, FEMS Immunol. Med. Mic. 10:47-54; Nilsson et al., 1998, J. Clin. Invest. 101:2640-2649; Josefsson et al., 2001, J. Infect. Dis. 184:1572-1580).

Information concerning S. aureus polypeptide sequences has been obtained from sequencing the S. aureus genome (Kuroda et al., 2001, Lancet 357:1225-1240; Baba et al., 2000, Lancet 359:1819-1827; Kunsch et al., European Patent Publication EP 0 786 519, published Jul. 30, 1997). To some extent, bioinformatics has been employed in efforts to characterize polypeptide sequences obtained from genome sequencing (see, e.g., EP 0 786 519, supra).

Techniques such as those involving display technology and sera from infected patients have been used in an effort to help identify genes coding for potential antigens (see, e.g., Foster et al., PCT International Publication no. WO 01/98499, published Dec. 27, 2001; Meinke et al., PCT International Publication no. WO 02/059148, published Aug. 1, 2002; Etz et al., 2002, Proc. Natl. Acad. Sci. USA 99:6573-6578).

### SUMMARY OF THE INVENTION

The present invention features polypeptides comprising an amino acid sequence structurally related to SEQ ID NO: 1 and uses of such polypeptides in the production of pharmaceutical compositions that provide a protective immune response against S. aureus infection. The amino acid sequence as set forth in SEQ ID NO: 1 represents the full-length protein sequence of a S. aureus antigen referred to herein as SACOL1902. A derivative of SEQ ID NO: 1 having the amino acid sequence as set forth in SEQ ID NO: 2, containing an NH₂-terminal histidine-tag (“his-tag”), was found to produce a protective immune response against S. aureus in animal models of S. aureus infection.

The present invention describes a polypeptide comprising an amino acid sequence having up to eight (8) amino acid alterations from the amino acid sequence as set forth in SEQ ID NO: 1. In one embodiment, the polypeptide does not consist of SEQ ID NO: 1 and/or SEQ ID NO: 6. The polypeptide can be used as an immunogen, wherein the reference to “immunogen” indicates the ability of that polypeptide to provide protective immunity against S. aureus, including but not limited to an S. aureus strain that expresses SEQ ID NO: 1.

Reference to “protective” immunity or immune response, when used in the context of a polypeptide, immunogen and/or treatment method described herein, indicates a detectable level of protection against S. aureus infection. This includes therapeutic and/or prophylactic measures reducing the likelihood of S. aureus infection or of obtaining a disorder(s) resulting from such infection, as well as reducing the severity of the infection and/or a disorder(s) resulting from such infection. As such, a protective immune response includes, for example, the ability to reduce bacterial load, ameliorate one or more disorders or symptoms associated with bacterial infection, and/or delaying the onset of disease progression resulting from S. aureus infection.

The level of protection can be assessed using animal models such as those described herein. For example, certain polypeptides described herein provide protection in a murine, lethal-challenge model and a rat, indwelling-catheter, sub-lethal challenge model.

A “disorder” is any condition resulting in whole or in part from S. aureus infection.

Reference to comprising an amino acid sequence with up to eight (8) amino acid alterations from the amino acid sequence as set forth in SEQ ID NO: 1 indicates that a SEQ ID NO: 1-related region is present and additional polypeptide regions may or may not be present. Each amino acid alteration is, independently, either an amino acid substitution, deletion, or addition.

Another aspect of the present invention describes an immunogen comprising a SEQ ID NO: 1-related polypeptide and one or more additional regions or moieties covalently joined to the polypeptide, wherein each region or moiety is independently selected from a region or moiety having at least one of the following properties: enhances the immune response, facilitates purification, or facilitates polypeptide stability. In one embodiment, the SEQ ID NO: 1-related polypeptide consists of an amino acid sequence with up to eight (8) amino acid alterations from the amino acid sequence as set forth in SEQ ID NO: 1. In a further embodiment, the SEQ ID NO: 1-related polypeptide consists of the amino acid sequence as set forth in SEQ ID NO: 1. The SEQ ID NO: 1-related polypeptide comprised within this immunogen provides protective immunity against S. aureus, including but not limited to a S. aureus strain that expresses SEQ ID NO: 1. The additional region or moiety can be, for example, an additional polypeptide region or a non-peptide region.

Reference to “purified” or “substantially purified” with regard to, for example, a polypeptide immunogen indicates presence of such polypeptide in an environment lacking one or more other polypeptides with which said polypeptide is naturally associated and/or represents at least about 10% of the total protein present.

Reference to “isolated” indicates a different form than found in nature. The different form can be, for example, a different purity than found in nature and/or a structure that is not found in nature. A structure not found in nature includes, for example, recombinant structures having different regions combined together.

The term “protein” or “polypeptide,” used interchangeably herein, indicates a contiguous amino acid sequence and does not provide a minimum or maximum size limitation. One or more amino acids present in the protein may contain a post-translational modification, such as glycosylation or disulfide bond formation.

Another aspect of the present invention describes a composition able to induce protective immunity against S. aureus in a patient. The composition comprises a pharmaceutically acceptable carrier and an immunologically effective amount of a polypeptide or immunogen described herein. Said polypeptide or immunogen may provide protective immunity against a S. aureus strain that expresses the polypeptide of SEQ ID NO: 1.

The term “immunologically effective amount” with regard to, for example, a polypeptide, immunogen, or composition thereof, means a sufficient amount such that, when introduced to a patient, produces an adequate level of the intended polypeptide or immunogen, resulting in an immune response against S. aureus. One skilled in the art recognizes that this level may vary. The amount should be sufficient to significantly prevent and/or reduce the likelihood or severity of a S. aureus infection.

Another aspect of the present invention describes a nucleic acid molecule comprising a recombinant gene which encodes a polypeptide that generates an immune response against S. aureus. A recombinant gene contains a recombinant nucleic acid molecule, wherein the nucleotide sequence of said nucleic acid molecule codes for a polypeptide along with regulatory elements for proper transcription and processing (which may include translational and post-translational elements). The recombinant gene can exist independent of a host genome or can be part of a host genome.

Such a nucleic acid molecule can be an expression vector. Preferably, the expression vector also contains an origin of replication for autonomous replication in a host cell, a selectable marker, a limited number of useful restriction enzyme sites and a potential for high copy number.

The term “nucleic acid” or “nucleic acid molecule” refers to ribonucleic acid (RNA) or deoxyribonucleic acid (DNA).

A recombinant nucleic acid molecule is a nucleic acid molecule that, by virtue of its sequence and/or form, does not occur in nature. Examples of recombinant nucleic acid molecules include purified nucleic acids, two or more nucleic acid regions combined together that provide a different nucleic acid than found in nature, and the absence of one or more nucleic acid regions (e.g., upstream or downstream regions) that are naturally associated with each other.

Further described herein are recombinant cells. Such recombinant cells comprise a recombinant gene encoding a polypeptide that provides a protective immune response against S. aureus. A recombinant cell can be used to make the polypeptide encoded by said recombinant gene, methods also described herein. The method involves growing a recombinant cell containing recombinant nucleic acid encoding the polypeptide and purifying the polypeptide.

Another aspect of the present invention describes a polypeptide that provides a protective immune response against S. aureus made by a process comprising the steps of growing a recombinant cell containing a recombinant nucleic acid molecule encoding the polypeptide in a host and purifying the polypeptide. Different host cells can be employed.

The present invention further provides methods of treating a patient against S. aureus infection. Said methods include inducing a protective immune response against S. aureus infection in a patient. The term “treatment” refers to both therapeutic treatment and prophylactic measures. Those in need of treatment include those already with an infection, as well as those prone to have an infection.

A further embodiment includes use of an immunologically effective amount of a SEQ ID NO: 1-related polypeptide, or immunogen thereof, in the manufacture of a medicament for inducing a protective immune response in a patient against S. aureus infection.

Unless particular terms are mutually exclusive, reference to “or” indicates either or both possibilities. Occasionally phrases such as “and/or” are used to highlight either or both possibilities.

Reference to open-ended terms such as “comprises” allows for additional elements or steps. Occasionally phrases such as “one or more” are used with or without open-ended terms to highlight the possibility of additional elements or steps.

Unless explicitly stated reference to terms such as “a,” “an,” or “the” is not limited to one and include plural reference unless the context clearly dictates otherwise. For example, “a cell” does not exclude “cells.” Occasionally phrases such as one or more are used to highlight the possible presence of a plurality.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### BRIEF DESCRIPTION OF THE DRAWINGS

FIG. 1 illustrates the amino acid sequence of SEQ ID NO: 2. The underlined portion represents a substantial portion of SEQ ID NO: 1, missing only the initiating methionine of SEQ ID NO: 1. The non-underlined region at the amino-terminal is a his-tag region.

FIG. 2 illustrates the amino acid sequence of SEQ ID NO: 1.

FIG. 3 illustrates a nucleic acid sequence (SEQ ID NO: 3) which encodes SEQ ID NO: 2. The portion encoding the amino-terminal his-tag is underlined.

FIG. 4A (experiment 1) and B (experiment 2) illustrate results from two challenge experiments using either a SEQ ID NO: 2 polypeptide (solid line) in aluminum hydroxyphosphate adjuvant or using adjuvant alone (dashed line).

### DETAILED DESCRIPTION OF THE INVENTION

The ability of SEQ ID NO: 1-related polypeptides to provide protective immunity against S. aureus infection is illustrated in the Examples provided below using SEQ ID NO: 2. SEQ ID NO: 2 is a derivative of SEQ ID NO: 1 containing an amino-terminal his-tag. The his-tag facilitates polypeptide purification and identification.

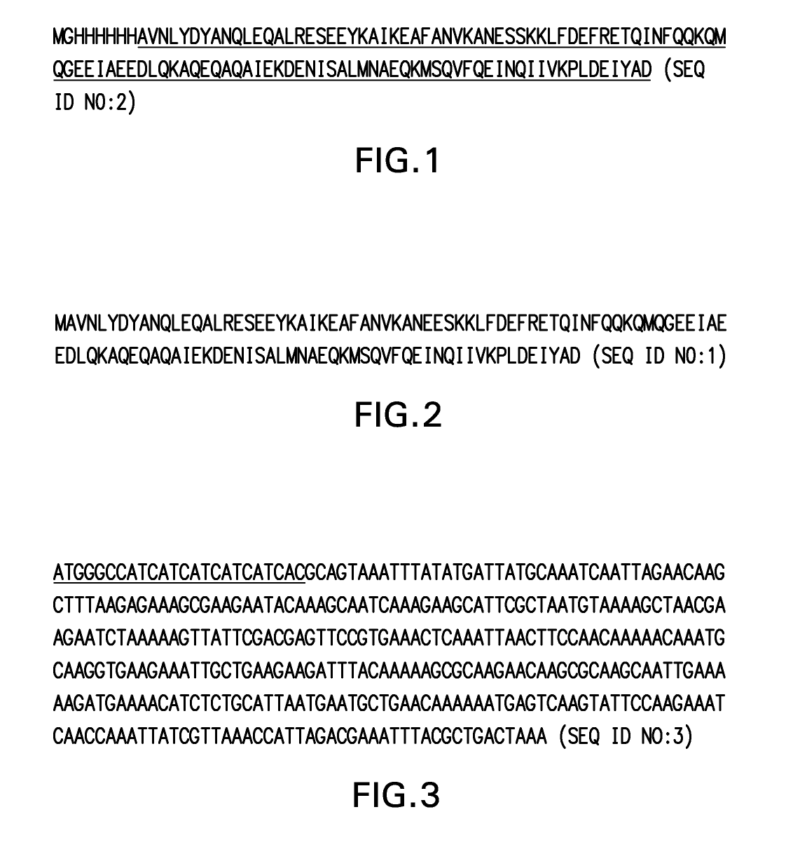

Polypeptides structurally related to SEQ ID NO: 1 include polypeptides containing corresponding regions present in different S. aureus strains and derivatives of naturally occurring regions. The amino acid sequence of SEQ ID NO: 1 is illustrated in FIG. 2. The relationship between the amino acid sequences as set forth in SEQ ID NOs: 1 and 2 is illustrated in FIG. 1.

### I. SACOL1902 (SEQ ID NO: 1) Sequences

S. aureus SACOL1902 is a conserved, surface-expressed protein. SACOL1902 has an amino acid sequence as set forth in SEQ ID NO: 1. This sequence is conserved among the thirteen S. aureus strains that have been sequenced thus far. Table 1 lists the thirteen S. aureus strains, their corresponding NCBI GenBank Accession nos. (both the revised versions and the original submission nos.), and the submitter for each genomic sequence.

TABLE 1

| | NCBI revised | | Original | |
|---|---|---|---|---|
| Strain | version | Locus Tag | submission | Submitted by |
| COL | NC_002951 | SACOL1902 | CP000046 | TIGR |
| N315 | NC_002745 | SA1663 | BA000018 | Kitasato Institute for Life |
| | | | | Science |
| Mu50 | NC_002758 | SAV1845 | BA000017 | Kitasato Institute for Life |
| | | | | Science |
| Mu3 | NC_009782 | SAHV_1830 | AP009351 | Juntendo University |
| | | | | School of Medicine |
| MW2 | NC_003923 | MW1786 | BA000033 | Nat'l Institute of Tech. & |
| | | | | Evaluation |
| NCTC | NC_007795 | SAOUHSC_01977 | CP000253 | University of Oklahoma |
| 8325 | | | | Health Sciences Center |
| Newman | NC_009782 | NWMN_1737 | AP009324 | Juntendo University |
| | | | | School of Medicine |
| MRSA252 | NC_002952 | SAR1936 | BX571856 | Sanger Institute |
| MSSA476 | NC_002953 | SAS1766 | BX571857 | Sanger Institute |
| JH1 | NC_009632 | SaurJN1_1932 | CP000736 | US DOE Joint Genome |
| | | | | Institute |
| JH9 | NC_009487 | SaurJH9_1898 | CP000703 | US DOE Joint Genome |
| | | | | Institute |
| USA300; | NC_007793 | SAUSA300_1795 | CP000255 | UCSF |
| FPR3757 | | | | |
| USA300_TCH1516 | NC_010079 | USA300HOU_1838 | CP000730 | Baylor |

PCT International Publication no. WO 00/50433 discloses a SACOL1902-related sequence, called Q13, having one amino acid difference from SEQ ID NO: 1 at residue position 5, set forth herein as SEQ ID NO: 6.

Other naturally occurring SACOL1902 sequences can be identified based on the presence of a high degree of sequence similarity or contiguous amino acids compared to a known SACOL1902 sequence. Contiguous amino acids provide characteristic tags. In different embodiments, a naturally occurring SACOL1902 sequence is a sequence found in a Staphylococcus, preferably S. aureus, having at least 20, at least 30, or at least 50 contiguous amino acids as in SEQ ID NO: 1; and/or having at least 93% sequence similarity or identity with SEQ ID NO: 1.

Percent sequence similarity (also referred to as percent identity) to a reference sequence can be determined by different algorithms and techniques well known in the art. Generally, sequence similarity is determined by first aligning the polypeptide sequence with the reference sequence to obtain maximum amino acid identity, allowing for gaps, additions and substitutions in one of the sequences, and then determining the number of identical amino acids in the corresponding regions. This number is divided by the total number of amino acids in the reference sequence (e.g., SEQ ID NO: 1), multiplied by 100, and rounded to the nearest whole number.

### II. SEQ ID NO: 1-Related Polypeptides

SEQ ID NO: 1-related polypeptides of the present invention contain an amino acid sequence that is at least 93% identical to SEQ ID NO: 1. Reference to “polypeptide” does not provide a minimum or maximum size limitation. The SEQ ID NO: 1-related polypeptides of the present invention provide protective immunity against S. aureus infection, including but not limited to a S. aureus strain that expresses SEQ ID NO: 1.

A polypeptide that contains eight (8) amino acid alterations from SEQ ID NO: 1 is approximately 93% identical to SEQ ID NO: 1. Each amino acid alteration is, independently, either an amino acid substitution, deletion, or addition. In different embodiments, the SEQ ID NO: 1-related polypeptide is at least 94%, at least 95%, at least 98%, or at least 99% identical to SEQ ID NO: 1; or differs from SEQ ID NO: 1 by 1, 2, 3, 4, 5, 6, 7, or 8 amino acid alterations.

In one embodiment, the SEQ ID NO: 1-related polypeptide is not SEQ ID NO: 1. In a further embodiment the SEQ ID NO: 1-related polypeptide is not SEQ ID NO: 6.

In another embodiment of the present invention, a polypeptide comprises or consists essentially of a SEQ ID NO: 1-related sequence that is between 93% and 98% identical to SEQ ID NO: 1. In this embodiment, said SEQ ID NO: 1-related sequence can have between 2 and 8 amino acid alterations from SEQ ID NO: 1.

In a still further embodiment of the present invention, a polypeptide immunogen comprises or consists essentially of a SEQ ID NO: 1-related sequence that is between 93% and 94% identical to SEQ ID NO: 1. In this embodiment, said SEQ ID NO: 1-related sequence can have 7 or 8 amino acid alterations from SEQ ID NO: 1.

Examples of said polypeptides of the present invention include polypeptides comprising or consisting essentially of the following amino acid portions of SEQ ID NO: 1: amino acids 5-110, amino acids 9-114, amino acids 1-106, amino acids 4-109, amino acids 8-113, amino acids 2-107, amino acids 3-108, amino acids 7-112, and amino acids 6-111. Additional amino acids that may be present include additional SEQ ID NO: 1 amino acids or other amino acid regions. A preferred additional amino acid is an amino-terminus methionine.

Reference to “consists essentially” of indicated amino acids indicates that the referred to amino acids are present and additional amino acids may be present. The additional amino acids can be at the carboxyl or amino terminus. In different embodiments 1, 2, 3, 4, 5, 6, 7 or 8 additional amino acids are present.

Alterations can be made to the SEQ ID NO: 1-related polypeptides described herein to obtain derivatives that induce protective immunity against S. aureus. Alterations can be performed, for example, to obtain a derivative that retains the ability to induce protective immunity against S. aureus or to obtain a derivative that, in addition to providing protective immunity, also has a region that can achieve a particular purpose.

Alterations can be made by taking into account both different SACOL1902 sequences and known properties of amino acids. Generally, when substituting different amino acids to retain activity, it is preferable to exchange amino acids having similar properties. Factors that can be taken into account for an amino acid substitution include amino acid size, charge, polarity, and hydrophobicity. For example, substituting a valine for leucine, an arginine for lysine, or an asparagine for glutamine represents good candidates for not inducing a change in polypeptide functioning. The effect of different amino acid R-groups on amino acid properties are well known in the art. (See, for example, Ausubel, Current Protocols in Molecular Biology, John Wiley, 1987-2002, Appendix 1C.)

Alterations to achieve a particular purpose include those designed to facilitate production or efficacy of the polypeptide; or cloning of the encoding nucleic acid. Polypeptide production can be facilitated through the use of an initiation codon (e.g., coding for methionine) suitable for recombinant expression. The methionine may be later removed during cellular processing. Cloning can be facilitated by, for example, the introduction of restriction sites which can be accompanied by amino acid additions or changes.

Efficacy of a polypeptide to induce a protective immune response can be improved through epitope enhancement. Epitope enhancement can be performed using different techniques such as those involving alteration of anchor residues to improve peptide affinity for MHC molecules and those that increase the affinity of the peptide-MHC complex for a T-cell receptor (Berzofsky et al., 2001, Nature Review 1:209-219).

Preferably, the polypeptide is a purified polypeptide. A “purified” polypeptide is present in an environment lacking one or more other polypeptides with which it is naturally associated and/or is represented by at least about 10% of the total protein present. In different embodiments, the purified polypeptide represents at least about 50%, at least about 75%, or at least about 95% of the total protein in a sample or preparation.

In an embodiment, the polypeptide is “substantially purified.” A substantially purified polypeptide is present in an environment lacking all, or most, other polypeptides with which the polypeptide is naturally associated. For example, a substantially purified S. aureus polypeptide is present in an environment lacking all, or most, other S. aureus polypeptides. An environment can be, for example, a sample or preparation.

Reference to “purified” or “substantially purified” does not require a polypeptide to undergo any purification and may include, for example, a chemically synthesized polypeptide that has not been purified.

Polypeptide stability can be enhanced by modifying the polypeptide carboxyl or amino terminus. Examples of possible modifications include amino terminus protecting groups such as acetyl, propyl, succinyl, benzyl, benzyloxycarbonyl or t-butyloxycarbonyl; and carboxyl terminus protecting groups such as amide, methylamide, and ethylamide.

In an embodiment of the present invention, a polypeptide described herein is part of an immunogen containing one or more additional regions or moieties covalently joined to the polypeptide, wherein each region or moiety is independently selected from a region or moiety having at least one of the following properties: enhances the immune response, facilitates purification, or facilitates polypeptide stability. Polypeptide stability can be enhanced, for example, using groups such as polyethylene glycol. Such additional regions or moieties can be covalently joined to the polypeptide through the carboxyl terminus, amino terminus or an internal region of the protein.

Polypeptide purification can be enhanced by adding a group to the carboxyl or amino terminus to facilitate purification. Examples of groups that can be used to facilitate purification include polypeptides providing affinity tags. Examples of affinity tags include a six-histidine-tag, trpE, glutathione and maltose-binding protein.

The ability of a polypeptide to produce an immune response can be improved using groups that generally enhance an immune response. Examples of groups that can be joined to a polypeptide to enhance an immune response against the polypeptide include cytokines such as IL-2 (Buchan et al., 2000, Molecular Immunology 37:545-552).

### III. Polypeptide Production

Polypeptides can be produced using standard techniques including those involving chemical synthesis and those involving purification from a cell producing the polypeptide. Techniques for chemical synthesis of polypeptides are well known in the art. (See e.g., Vincent, Peptide and Protein Drug Delivery, New York, N.Y., Decker, 1990.) Techniques for recombinant polypeptide production and purification are also well known in the art. (See, e.g., Ausubel, Current Protocols in Molecular Biology, John Wiley, 1987-2002.)

Obtaining polypeptides from a cell is facilitated by using recombinant nucleic acid techniques to produce the polypeptide. Recombinant nucleic acid techniques for producing a polypeptide involve introducing, or producing, a recombinant gene encoding the polypeptide in a cell and expressing the polypeptide.

A recombinant gene contains a nucleic acid that encodes a polypeptide, along with regulatory elements for polypeptide expression. The recombinant gene can be present in a cellular genome or can be part of an expression vector.

The regulatory elements that may be present as part of a recombinant gene include those naturally associated with the polypeptide-encoding sequence, as well as exogenous regulatory elements not naturally associated with the polypeptide-encoding sequence. Exogenous regulatory elements, such as an exogenous promoter, can be useful for expressing a recombinant gene in a particular host or for increasing the level of expression. Generally, the regulatory elements that are present in a recombinant gene include a transcriptional promoter, a ribosome binding site, a transcriptional terminator, and an optionally present operator. A preferred element for processing in eukaryotic cells is a polyadenylation signal.

Expression of a recombinant gene in a cell is facilitated through the use of an expression vector. In addition to a recombinant gene, an expression vector usually contains an origin of replication for autonomous replication in a host cell, a selectable marker, a limited number of useful restriction enzyme sites, and a potential for high copy number. Examples of expression vectors are cloning vectors, modified cloning vectors, specifically designed plasmids and viruses.

Due to the degeneracy of the genetic code, a large number of different encoding nucleic acid sequences can be used to code for a particular polypeptide. The degeneracy of the genetic code arises because almost all amino acids are encoded by different combinations of nucleotide triplets or “codons.” Naturally occurring amino acids are encoded by codons as follows:

A=Ala=Alanine: codons GCA, GCC, GCG, GCU
C=Cys=Cysteine: codons UGC, UGU
D=Asp=Aspartic acid: codons GAC, GAU
E=Glu=Glutamic acid: codons GAA, GAG
F=Phe=Phenylalanine: codons UUC, UUU
G=Gly=Glycine: codons GGA, GGC, GGG, GGU
H=His=Histidine: codons CAC, CAU
I=Ile=Isoleucine: codons AUA, AUC, AUU
K=Lys=Lysine: codons AAA, AAG
L=Leu=Leucine: codons UUA, UUG, CUA, CUC, CUG, CUU
M=Met=Methionine: codon AUG
N=Asn=Asparagine: codons AAC, AAU
P=Pro=Proline: codons CCA, CCC, CCG, CCU
Q=Gln=Glutamine: codons CAA, CAG
R=Arg=Arginine: codons AGA, AGG, CGA, CGC, CGG, CGU
S=Ser=Serine: codons AGC, AGU, UCA, UCC, UCG, UCU
T=Thr=Threonine: codons ACA, ACC, ACG, ACU
V=Val=Valine: codons QUA, GUC, GUG, GUU
W=Trp=Tryptophan: codon UGG
Y=Tyr=Tyrosine: codons UAC, UAU

Suitable cells for recombinant nucleic acid expression of SEQ ID NO: 1-related polypeptides are prokaryotes and eukaryotes. Examples of prokaryotic cells include E. coli; members of the Staphylococcus genus, such as S. aureus and S. epidermidis; members of the Lactobacillus genus, such as L. plantarum; members of the Lactococcus genus, such as L. lactis; members of the Bacillus genus, such as B. subtilis; members of the Corynebacterium genus such as C. glutamicum; and members of the Pseudomonas genus such as Ps. fluorescens. Examples of eukaryotic cells include mammalian cells; insect cells; and yeast cells, such as members of the Saccharomyces genus (e.g., S. cerevisiae), members of the Pichia genus (e.g., P. pastoris), members of the Hansenula genus (e.g., H. polymorpha), members of the Kluyveromyces genus (e.g., K. lactis or K. fragilis) and members of the Schizosaccharomyces genus (e.g., S. pombe).

Techniques for recombinant gene production, introduction into a cell, and recombinant gene expression are well known in the art. Examples of such techniques are provided in references such as Ausubel, Current Protocols in Molecular Biology, John Wiley, 1987-2002; and Sambrook et al., Molecular Cloning, A Laboratory Manual, 2^{nd} Edition, Cold Spring Harbor Laboratory Press, 1989.

If desired, expression in a particular host can be enhanced through codon optimization. Codon optimization includes use of more preferred codons. Techniques for codon optimization in different hosts are well known in the art.

SEQ ID NO: 1-related polypeptides may contain post translational modifications, for example, N-linked glycosylation, O-linked glycosylation, or acetylation. Reference to “polypeptide” or an amino acid sequence of a polypeptide includes polypeptides containing one or more amino acids having a structure of a post-translational modification from a host cell, such as a yeast host.

Post translational modifications can be produced chemically or by making use of suitable hosts. For example, in S. cerevisiae the nature of the penultimate amino acid appears to determine whether the N-terminal methionine is removed. Furthermore, the nature of the penultimate amino acid also determines whether the N-terminal amino acid is N^{α}-acetylated (Huang et al., 1987, Biochemistry 26: 8242-8246). Another example includes a polypeptide targeted for secretion due to the presence of a secretory leader (e.g., signal peptide), where the protein is modified by N-linked or O-linked glycosylation (Kukuruzinska et al., 1987, Ann. Rev. Biochem. 56:915-944).

### IV. Adjuvants

Adjuvants are substances that can assist an immunogen (e.g., a polypeptide, pharmaceutical composition containing a polypeptide) in producing an immune response. Adjuvants can function by different mechanisms such as one or more of the following: increasing the antigen biologic or immunologic half-life; improving antigen delivery to antigen-presenting cells; improving antigen processing and presentation by antigen-presenting cells; and, inducing production of immunomodulatory cytokines (Vogel, Clinical Infectious Diseases 30(suppl. 3):S266-270, 2000). In one embodiment of the present invention, an adjuvant is used.

A variety of different types of adjuvants can be employed to assist in the production of an immune response. Examples of particular adjuvants include aluminum hydroxide; aluminum phosphate, or other salts of aluminum; calcium phosphate; DNA CpG motifs; monophosphoryl lipid A; cholera toxin; E. coli heat-labile toxin; pertussis toxin; muramyl dipeptide; Freund's incomplete adjuvant; MF59; SAF; immunostimulatory complexes; liposomes; biodegradable microspheres; saponins; nonionic block copolymers; muramyl peptide analogues; polyphosphazene; synthetic polynucleotides; IFN-γ; IL-2; IL-12; and ISCOMS. (Vogel, Clinical Infectious Diseases 30(suppl 3):S266-270, 2000; Klein et al., 2000, Journal of Pharmaceutical Sciences 89:311-321; Rimmelzwaan et al., 2001, Vaccine 19:1180-1187; Kersten, 2003, Vaccine 21:915-920; O'Hagen, 2001, Curr. Drug Target Infect. Disord. 1:273-286.)

### V. Patients for Inducing Protective Immunity

A “patient” refers to a mammal capable of being infected with S. aureus. In one embodiment, a patient is a human. A patient can be treated prophylactically or therapeutically. Prophylactic treatment provides sufficient protective immunity to reduce the likelihood, or severity, of an S. aureus infection. Therapeutic treatment can be performed to reduce the severity of an S. aureus infection.

Prophylactic treatment can be performed using a pharmaceutical composition containing a polypeptide or immunogen described herein. Such treatment is preferably performed on a human. Pharmaceutical compositions can be administered to the general population or to those persons at an increased risk of S. aureus infection.

Those in need of treatment include those already with an infection, as well as those prone to have an infection or in which the likelihood of an infection is to be reduced. Persons with an increased risk of S. aureus infection include health care workers; hospital patients; patients with a weakened immune system; patients undergoing surgery; patients receiving foreign body implants, such a catheter or a vascular device; patients facing therapy leading to a weakened immunity; patients under diagnostic procedures involving foreign bodies; and, persons in professions having an increased risk of burn or wound injury.

Foreign bodies used in diagnostic or therapeutic procedures include indwelling catheters or implanted polymer devices. Examples of foreign body-associated S. aureus infections include septicemia/endocarditis (e.g., intravascular catheters, vascular prostheses, pacemaker leads, defibrillator systems, prosthetic heart valves, and left ventricular assist devices); peritonitis (e.g., ventriculo-peritoneal cerebrospinal fluid (CSF) shunts and continuous ambulatory peritoneal dialysis catheter systems); ventriculitis (e.g., internal and external CSF shunts); and chronic polymer-associated syndromes (e.g., prosthetic joint/hip loosening, fibrous capsular contracture syndrome after mammary argumentation with silicone prosthesis and late-onset endophtalmisis after implantation of artificial intraocular lenses following cataract surgery). (See, Hellmann and Peters, Biology and Pathogenicity of Staphylococcus epidermidis, In: Gram Positive Pathogens, Eds. Fischetti et al., American Society for Microbiology, Washington D.C. 2000.)

Non-human patients that can be infected with S. aureus include cows, pigs, sheep, goats, rabbits, horses, dogs, cats, rats and mice. Treatment of non-human patients is useful in both protecting pets and livestock and evaluating the efficacy of a particular treatment.

In an embodiment, a patient is treated prophylactically in conjunction with a therapeutic or medical procedure involving a foreign body. In additional embodiments, the patient is immunized at about 1 month, about 2 month or about 2-6 months prior to the procedure.

An embodiment also includes one or more of the polypeptide immunogens or compositions thereof, described herein, or a vaccine comprising or consisting of said immunogens or compositions (i) for use in, (ii) for use as a medicament for, or (iii) for use in the preparation of a medicament for: (a) therapy (e.g., of the human body); (b) medicine; (c) inhibition of S. aureus replication; (d) treatment or prophylaxis of infection by S. aureus; or, (e) treatment, prophylaxis of, or delay in the onset or progression of S. aureus-associated disease(s). In these uses, the polypeptide immunogens, compositions thereof, and/or vaccines comprising or consisting of said immunogens or compositions can optionally be employed in combination with one or more anti-bacterial agents (e.g., anti-bacterial compounds; combination vaccines, described infra).

### VI. Combination Vaccines

SEQ ID NO: 1-related polypeptides can be used alone or in combination with other immunogens to induce an immune response. Additional immunogens that may be present include one or more additional S. aureus immunogens, one or more immunogens targeting one or more other Staphylococcus organisms such as S. epidermidis, S. haemolyticus, S. warneri, or S. lugunensi, and/or one or more immunogens targeting other infections organisms.

Examples of one or more additional immunogens include ORF0657n-related polypeptides (Anderson et al., International Publication no. WO 05/009379); ORF0657/ORF0190 hybrid polypeptides (Anderson et al., International Publication no. WO 05/009378); sai-1-related polypeptides (Anderson et al., International Publication no. WO 05/79315); ORF0594-related polypeptides (Anderson et al., International Publication no. WO 05/086663); ORF0826-related polypeptides (Anderson et al., International Publication no. WO 05/115113); PBP4-related polypeptides (Anderson et al., International Publication no. WO 06/033918); AhpC-related polypeptides and AhpC-AhpF compositions (Kelly et al. International Publication No. WO 06/078680); S. aureus type 5 and type 8 capsular polysaccharides (Shinefield et al., 2002, N. Eng. J. Med. 346:491-496); collagen adhesin, fibrinogen binding proteins, and clumping factor (Mamo et al., 199, FEMS Immunol. Med. Microbiol. 10:47-54; Nilsson et al., 1998, J. Clin. Invest. 101:2640-2649; Josefsson et al., 2001, J. of Infect. Dis. 184:1572-1580); and polysaccharide intercellular adhesin and fragments thereof (Joyce et al., 2003, Carbohydrate Research 338:903-922).

### VII. Administration

The SEQ ID NO: 1-related polypeptides and immunogens described herein can be formulated and administered to a patient using the guidance provided herein along with techniques well known in the art. Guidelines for pharmaceutical administration in general are provided in, for example, Vaccines Eds. Plotkin and Orenstein, W.B. Sanders Company, 1999; Remington's Pharmaceutical Sciences 20^{th} Edition, Ed. Gennaro, Mack Publishing, 2000; and Modern Pharmaceutics 2^{nd} Edition, Eds. Banker and Rhodes, Marcel Dekker, Inc., 1990.

Pharmaceutically acceptable carriers facilitate storage and administration of an immunogen to a patient. Pharmaceutically acceptable carriers may contain different components such as a buffer, sterile water for injection, normal saline or phosphate-buffered saline, sucrose, histidine, salts and polysorbate. As such, the present invention encompasses compositions able to induce a protective immune response in a patient against S. aureus infection comprising an immunologically effective amount of a SEQ ID NO: 1-related polypeptide, or immunogen thereof, and a pharmaceutically acceptable carrier. The composition may further comprise an adjuvant.

Immunogens can be administered by different routes such as subcutaneous, intramuscular, or mucosal. Subcutaneous and intramuscular administration can be performed using, for example, needles or jet-injectors.

Suitable dosing regimens are preferably determined taking into account factors well known in the art including age, weight, sex and medical condition of the patient; the route of administration; the desired effect; and the particular compound employed. The immunogen can be used in multi-dose vaccine formats. It is expected that a dose would consist of the range of 1.0 μg to 1.0 mg total polypeptide. In different embodiments of the present invention, the dosage range is from 5.0 μg to 500 μg, 0.01 mg to 1.0 mg, or 0.1 mg to 1.0 mg.

The timing of doses depends upon factors well known in the art. After the initial administration, one or more additional doses may be administered to maintain and/or boost antibody titers. An example of a dosing regime would be day 1, 1 month, a third dose at either 4, 6 or 12 months, and additional booster doses at distant times as needed.

### VIII. Generation of Antibodies

A SEQ ID NO: 1-related polypeptide can be used to generate antibodies and antibody fragments binding to the polypeptide or to S. aureus. Such antibodies and antibody fragments have different uses including use in polypeptide purification, S. aureus identification, or in therapeutic or prophylactic treatment against S. aureus infection.

Antibodies can be polyclonal or monoclonal. Techniques for producing and using antibodies, including human antibodies, are well known in the art (see, e.g., Ausubel, Current Protocols in Molecular Biology, John Wiley, 1987-2002; Harlow et al., Antibodies, A Laboratory Manual, Cold Spring Harbor Laboratory, 1988; Kohler et al., 1975, Nature 256:495-497; Azzazy et al., 2002, Clinical Biochem. 35:425-445; Berger et al., 2002, Am. J. Med. Sci. 324:14-40).

Proper glycosylation can be important for antibody function (Yoo et al., 2002, J. Immunol. Methods 261:1-20; Li et al., 2006, Nature Biotechno. 24:210-215). Naturally occurring antibodies contain at least one N-linked carbohydrate attached to a heavy chain (Yoo et al., supra). Additional N-linked carbohydrates and O-linked carbohydrates may be present and may be important for antibody function. Id.

Different types of host cells can be used to provide for efficient post-translational modifications including mammalian host cells and non-mammalian cells. Examples of mammalian host cells include Chinese hamster ovary (Cho), HeLa, C6, PC12, and myeloma cells (Yoo et al., supra; Persic et al., 1997, Gene 187:9-18). Non-mammalian cells can be modified to replicate human glycosylation (Li et al., supra). Glycoengineered Pichia pastoris is an example of such a modified non-mammalian cell (Li et al., supra).

### IX. Nucleic Acid Vaccine

Nucleic acid encoding a SEQ ID NO: 1-related polypeptide can be introduced into a patient using vectors suitable for therapeutic administration. Suitable vectors can deliver nucleic acid into a target cell without causing an unacceptable side effect. Examples of vectors that can be employed include plasmid vectors and viral based vectors. (Barouch, 2006, J. Pathol. 208:283-289; Emini et al., International Publication no. WO 03/031588.)

Cellular expression is achieved using a gene expression cassette encoding a desired polypeptide. The gene expression cassette contains regulatory elements for producing and processing a sufficient amount of nucleic acid inside a target cell to achieve a beneficial effect.

Examples of viral vectors include first and second generation adenovectors, helper dependent adenovectors, adeno-associated viral vectors, retroviral vectors, alphavirus vectors (e.g., Venezuelan Equine Encephalitis virus vectors), and plasmid vectors. (Hitt et al., 1997, Advances in Pharmacology 40:137-206; Johnston et al., U.S. Pat. No. 6,156,588; Johnston et al., International PCT Publication no. WO 95/32733; Barouch, 2006, J. Pathol. 208:283-289; Emini et al., International PCT Publication no. WO 03/031588.)

Adenovectors can be based on different adenovirus serotypes such as those found in humans or animals. Examples of animal adenoviruses include bovine, porcine, chimpanzee, murine, canine, and avian (CELO). (Emini et al., International PCT Publication no. WO 03/031588; Colloca et al., International PCT Publication no. WO 05/071093.) Human adenovirus include Group B, C, D, or E serotypes such as type 2 (“Ad2”), 4 (“Ad4”), 5 (“Ad5”), 6 (“Ad6”), 24 (“Ad24”), 26 (“Ad26”), 34 (“Ad34”) and 35 (“Ad35”).

Nucleic acid vaccines can be administered using different techniques and dosing regimes. (Emini et al., International PCT Publication no. WO 03/031588.) For example, the vaccine can be administered intramuscular by injection with or without one or more electric pulses. Electric mediated transfer can assist genetic immunization by stimulating both humoral and cellular immune responses. Examples of dosing regimes include prime-boost and heterologous prime-boost approaches. (Emini et al., International PCT Publication no. WO 03/031588.)

All publications mentioned herein are incorporated by reference for the purpose of describing and disclosing methodologies and materials that might be used in connection with the present invention. Nothing herein is to be construed as an admission that the invention is not entitled to antedate such disclosure by virtue of prior invention.

Having described preferred embodiments of the invention with reference to the accompanying drawings, it is to be understood that the invention is not limited to those precise embodiments, and that various changes and modifications may be effected therein by one skilled in the art without departing from the scope or spirit of the invention as defined in the appended claims. Thus, the following examples illustrate, but do not limit, the invention.

### Example 1

### Protective Immunity

This example illustrates the ability of SEQ ID NO: 1-related polypeptides to provide protective immunity in an animal model. SEQ ID NO: 2, a His-tagged derivative of SEQ ID NO: 1, was shown to provide protective immunity.

SEQ ID NO 2 cloning and expression—The protein encoded by the SACOL1902 gene was designed to be expressed from the pETBlue-1 vector (Novagen, Madison, Wis.) with the N-terminal histidine residues and the stop codon encoded by the vector. In addition, a glycine residue was added to the protein after the methionine initiator. PCR primers were designed to amplify SACOL1902 starting at the first methionine codon and ending prior to the stop codon at the terminal glutamate residue. The forward and reverse primers were: 5′-ATGGGCCATCATCATCATCATCACGCAGTAAATTTATATGATTATGCAAATCAATTA G-3′ (SEQ ID NO: 5) and 5′-TTAGTCAGCGTAAATTTCGTC-3′ (SEQ ID NO: 4), respectively. Genomic DNA was purified from S. aureus strain COL, using a Wizard® Genomic DNA Purification kit (Promega, Madison, Wis.) according to manufacturer's instructions. This genomic DNA was used as the template for the PCR reaction

The SACOL1902 gene was amplified by PCR in a 50 μL volume reaction containing 250 ng genomic DNA, 125 ng each forward and reverse primer, 1 microliter 50 mM dNTPs, 2.5 units of taq polymerase and 1× buffer (Clontech advantage cDNA kit). The thermacycling conditions were as follows: one cycle of 94° C. for 1 min; 32 cycles of 94° C. for 1 min, 53° C. for 30 seconds, 68° C. for two min; one cycle of 68° C. for 4 min. The amplified DNA sequence (367 bp) was ligated into the pETBlue-1 linear vector by using the AccepTor vector kit (Novagen). The ligation reaction was transformed into competent NovaBlue Single™. The transformation mixture was grown overnight at 37° C. on LB (Luria-Bertani) agar plates containing 50 μg/mL cabenicillin, 12.5 μg/mL tetracycline, 40 μg/mL X-Gal and 20 μL of 100 mM IPTG. White colonies were selected and grown in Luria Broth (LB) with 50 μg/mL ampicillin. DNA minipreps were made (Qiagen), and the appropriate insert was determined by restriction endonuclease digestion. The plasmid DNA was sequenced, and a clone containing no DNA changes from the desired sequence was selected and designated COLSA1902 #4.

E. coli Tuner (DE3) pLacI competent cells were transformed with COLSA1902 #4 and grown on LB plates containing ampicillin (100 μg/mL) and chloramphenicol (34 ng/ml). To test for expression of SACOL1902, an isolated colony was inoculated into 5 mL of liquid LB, 1% glucose, 100 μg/mL ampicillin and incubated at 37° C., 250 rpm until OD₆₀₀ was between 0.5 to 1.0. Induction of expression was performed by adding IPTG (final IPTG concentration of 0.4 mM) and incubated at 37° C. for 3 hours. For lysate preparation, 1.0 mL culture volume from uninduced and induced cultures, respectively, were collected by centrifugation and resuspended in 300 μL of BugBuster HT (EMD Sciences, Madison, Wis.) and 3 μL Proteinase Inhibitor Cocktail (Sigma, St. Louis, Mo.). The mixtures were held on ice for 5 minutes and subsequently sonicated three times for ten seconds, each with cooling in between. To obtain “soluble” and “insoluble” fractions the mixture was centrifuged at 13,000 rpm for fifteen minutes at 4° C. The supernatant was designated “soluble” and the pellet was resuspended in 300 μL of BugBuster HT and 3 μL Proteinase of Inhibitor Cocktail and designated “insoluble.”

For analysis of expression of his-tagged SACOL1902 (encoded by SEQ ID NO: 2) by Coomassie staining of SDS-PAGE gels, samples were subjected to electrophoresis on 4-12% gradient NuPage Bis-Tris gels (Invitrogen) in 1×MES SDS buffer (Invitrogen) under reducing and denaturing conditions. To estimate protein size, standards between 6 and 188 kDa (Invitrogen) were run in parallel with the lysates. The gels were stained with Bio-Safe Coomassie, a Coomassie G250 stain (BIO-RAD) according to the manufacturer's protocol. Western blot was performed and the signal was detected by anti-His mAb (EMD Sciences)

A 14.2-kDa protein was specifically detected by both Coomasie staining and Western blot in lysates. Good expression was obtained with SACOL1902 localizing to the soluble fraction.

SEQ ID NO: 2 purification—Direct scale-up of the above small scale procedure into stirred tank fermenters (30 liter scale) with a 20 liter working volume was achieved. Inoculum was cultivated in a 250 mL flask containing 50 mL of Luria-Bertani (LB) medium (plus ampicillin) and inoculated with 1 mL of frozen seed culture and cultivated for 6 hours. One mL of this seed was used to inoculate a 2 liter flask containing 500 mL of LB medium (plus ampicillin) and incubated for 16 hours. A large scale fermenter (30 liter scale) was cultivated with 20 liters of LB medium (plus ampicillin). The fermentation parameters of the fermenter were: pressure=5 psig, agitation speed=300 rpms, airflow=7.5 liters/minute and temperature=37° C. Cells were incubated to an optical density (OD) of 1.3 optical density units, at a wavelength of 600 nm, and induced with Isopropyl-β-K-Thiogalactoside (IPTG) at a concentration of 1 mM. Induction time with IPTG was two hours. Cells were harvested by lowering the temperature to 15° C., concentrated by passage through a 500KMWCO hollow fiber cartridge, and centrifuged at 8,000 times gravity at 4° C. for 20 minutes. Supernatants were decanted and the recombinant E. coli wet cell pellets were frozen at −70° C.

Frozen recombinant E. coli cell paste (24 grams) was thawed and resuspended in two volumes of Lysis Buffer (50 mM sodium phosphate, pH 8.0, 0.15 M NaCl, 2 mM magnesium chloride, 10 mM imidazole, 20 mM 2-mercaptoethanol, 0.1% Tween-80, and protease inhibitor cocktail (Complete™, EDTA-Free, Roche #1873580-one tablet per 50 mL Lysis Buffer). Benzonase (EM #1.01697.0002) was added to the cell suspension at 125 Units/mL). A lysate was prepared with a microfluidizer. The Lysate was stirred for three hours at 4° C., and was clarified by centrifugation at 10,000×g for 10 minutes at 4° C. The supernatant was filtered through a glass-fiber pre-filter Millipore and NaCl was added to a final concentration of 0.5 M from a 5 M stock solution. The Filtered Supernatant was added to Ni-NTA agarose chromatography resin (Qiagen #30250) and the slurry was mixed overnight at 4° C. The slurry of chromatography resin was poured into a chromatography column, and the non-bound fraction was collected by gravity from the column outlet. The column was washed with ten column volumes of Wash Buffer (50 mM sodium phosphate, pH 8.0, 0.5 M NaCl, 2 mM magnesium chloride, 10 mM imidazole, 20 mM 2-mercaptoethanol, 0.1% Tween-80, and protease inhibitor cocktail (Complete™, EDTA-Free, Roche #1873580-one tablet per 50 mL Wash Buffer)). The column was eluted with Elution Buffer (50 mM sodium phosphate, pH 7.4, 0.3 M imidazole, 2 mM magnesium chloride, 0.1% Tween-80, and 20 mM 2-mercaptoethanol). Fractions containing protein were identified by dot blot on nitrocellulose membrane with Ponceau-S staining, and fractions containing the highest protein concentrations were pooled to make the Ni-IMAC Product. The Ni-IMAC Product was fractionated by SEC. SEC fractions containing the product protein were identified by SDS/PAGE with Coomassie staining. Product-containing SEC fractions were pooled to make the SEC Product. The SEC Product was sterile-filtered and adsorbed on aluminum hydroxyphosphate adjuvant at a final concentration of 0.2 mg/mL.

Preparation of S. aureus challenge—S. aureus strain Becker was grown on TSA plates at 37° C. overnight. The bacteria were washed from the TSA plates by adding 5 mL of PBS onto a plate and gently resuspending the bacteria with a sterile spreader. The bacterial suspension was spun at 6000 rpm for 20 minutes using a Sorvall RC-5B centrifuge (DuPont Instruments). The pellet was resuspended in 16% glycerol and aliquots were stored frozen at −70° C.

Prior to use, inocula were thawed, appropriately diluted, and used for infection. Each stock was titrated to determine the lethal dose in mice. The potency of the bacterial inoculum (80 to 90% lethality) was constantly monitored to assure reproducibility of the model.

Protection studies for a SEQ ID NO: 2 polypeptide in a murine, lethal-challenge model—In two independent experiments, twenty BALB/c mice each were immunized with three doses of SEQ ID NO: 2 polypeptide (20 μg per injection) on aluminum hydroxyphosphate adjuvant (450 μg per injection). Aluminum hydroxyphosphate adjuvant (AHP) is described by Klein et al. (2000, J. Pharm. Sci. 89: 311-321). The materials were administered as two 50 μL intramuscular injections on days 0, 7 and 21. The mice were bled on day 28, and their sera were screened by ELISA for reactivity to SEQ ID NO: 2. Twenty mice each were injected with phosphate buffered saline (PBS) as a control group.

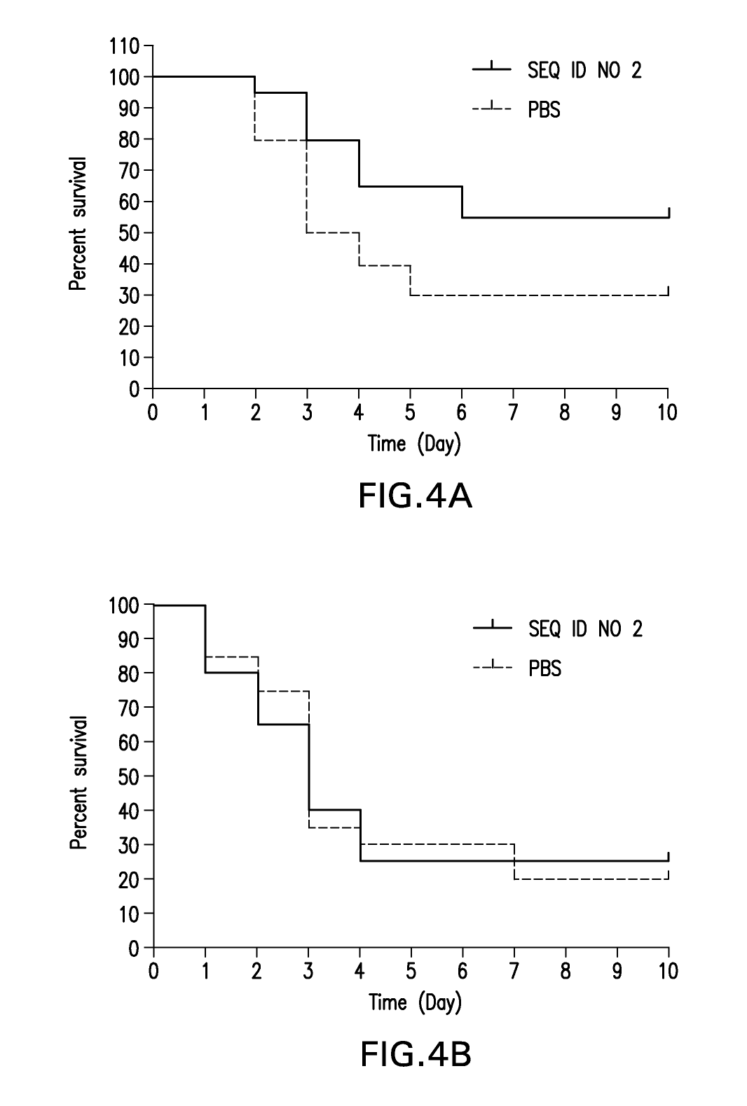

On day 35 of each experiment the mice were challenged by intravenous injection of S. aureus (dose 7×10⁸ CFU/mL). The mice were monitored over a 10 day period for survival. At the end of the first experiment, 11 mice survived in the SEQ ID NO: 2 polypeptide-immunized group, compared to 3 surviving in the PBS control group. The results are illustrated in FIG. 4A. In the second experiment 5 mice survived in the SEQ ID NO: 2 polypeptide-immunized group, compared to 4 surviving in the PBS control group. The results are illustrated in FIG. 4B.

Protection studies for a SEQ ID NO: 2 polypeptide in a rat, indwelling-catheter model—To assess whether active immunization against SEQ ID NO: 2 can prevent S. aureus infection of implanted devices, a rat indwelling catheter model was used. Sprague-Dawley rats, 3-4 weeks of age, were immunized on Day 0, 7 and 21 intraperitoneally with three doses of SEQ ID NO: 2 polypeptide (20 μg per injection) on aluminum hydroxyphosphate adjuvant (450 μg per injection), and 10 rats each were injected with aluminum hydroxyphosphate adjuvant (450 mg per injection). Aluminum hydroxyphosphate adjuvant (AHP) is described by Klein et al. (supra). The materials were administered as a single 100 μL intraperitoneal injection. The rats were bled on day 28, and their sera were screened by ELISA for reactivity to SEQ ID NO: 2. On Day 35, the animals underwent surgery to place an indwelling catheter into the jugular vein. The animals were rested for approximately 10 days after surgery, at which time a sub-lethal challenge of S. aureus strain Becker (2-7×10⁹ CFU) was given intravenously via the tail vein. The rats were sacrificed 24 hours post challenge, and the catheters were removed. The presence of S. aureus bacteria on the catheters was assessed by culturing the entire catheter on mannitol salt agar plates. If any sign of S. aureus outgrowth was observed on the plate, the catheter was scored as culture positive. After two independent experiments (a total of 20 immunized rats), 8 of 20 catheters were culture positive (40%). Whereas, 20 of 20 catheters were culture positive in the control rats (100%). The results are listed in Table 2.

TABLE 2
Protection of indwelling catheters from S. aureus colonization

| | |
|---|---|
| Number of | |
| culture | Total |
| positive catheters | number of culture |

| Treatment | Exp #1 | Exp #2 | positive catheters (%) |
|---|---|---|---|
| SEQ ID NO: 2 | 6/10 | 2/10 | 8/20 (40%) |
| AHP | 10/10 | 10/10 | 20/20 (100%) |

Other embodiments are within the following claims. While several embodiments have been shown and described, various modifications may be made without departing from the spirit and scope of the present invention.

1. A polypeptide comprising an amino acid sequence with up to 8 amino acid alterations from the amino acid sequence as set forth in SEQ ID NO: 1, wherein said polypeptide is not SEQ ID NO: 1 or SEQ ID NO: 6, and wherein said polypeptide provides protective immunity against S. aureus.

2. The polypeptide of claim 1, wherein said polypeptide comprises a portion of SEQ ID NO: 1 selected from the group consisting of: amino acids 5-110, amino acids 9-114, amino acids 1-106, amino acids 4-109, amino acids 8-113, amino acids 2-107, amino acids 3-108, amino acids 7-112, and amino acids 6-111.

3. The polypeptide of claim 1, wherein said polypeptide is substantially purified.

4. The polypeptide of claim 3, wherein said polypeptide provides protective immunity against a S. aureus strain that expresses SEQ ID NO: 1.

5. An immunogen comprising a polypeptide consisting of an amino acid sequence with up to 8 amino acid alterations from SEQ ID NO: 1 and one or more additional regions or moieties covalently joined to said amino acid sequence, wherein each region or moiety is independently selected from a region or moiety having at least one of the following properties: enhances the immune response, facilitates purification, or facilitates polypeptide stability.

6. The immunogen of claim 5, wherein said polypeptide provides protective immunity against a S. aureus strain that expresses SEQ ID NO: 1.

7. A composition able to induce a protective immune response in a patient against S. aureus infection comprising an immunologically effective amount of
the polypeptide of claim 4
and a pharmaceutically acceptable carrier.

8. A composition able to induce a protective immune response in a patient against S. aureus infection comprising an immunologically effective amount of a polypeptide comprising an amino acid sequence with up to 8 amino acid alterations from the amino acid sequence as set forth in SEQ ID NO: 1, wherein said polypeptide does not consist of the amino acid sequence as set forth in SEQ ID NO: 1, and a pharmaceutically acceptable carrier.

9. The composition of claim 8, wherein said composition provides protective immunity against a S. aureus strain that expresses SEQ ID NO: 1.

10. The composition of claim 8, wherein said composition further comprises an adjuvant.

11. A nucleic acid molecule comprising a recombinant gene which comprises a nucleotide sequence encoding the polypeptide of claim 1.

12. The nucleic acid molecule of claim 11, wherein said nucleic acid molecule is an expression vector.

13. A recombinant cell comprising a recombinant gene which comprises a nucleotide sequence encoding the polypeptide of claim 1.

14. A method of making a polypeptide comprising the steps of:
(a) growing the recombinant cell of claim 13 under conditions wherein said polypeptide is expressed; and,
(b) purifying said polypeptide.

15. A method of inducing a protective immune response in a patient against a S. aureus infection comprising the step of administering to said patient an immunologically effective amount of one or more of the following:
(a) the polypeptide of claim 1;
(b) a polypeptide having the amino acid sequence as set forth in SEQ ID NO: 1; or
(c) a polypeptide having the amino acid sequence as set forth in SEQ ID NO: 6.

16. The method of claim 15, wherein said patient is a human.

17-19. (canceled)

20. A composition able to induce a protective immune response in a patient against S. aureus infection comprising an immunologically effective amount of the immunogen of claim 5 and a pharmaceutically acceptable carrier.

21. A method of inducing a protective immune response in a patient against a S. aureus infection comprising the step of administering to said patient an immunologically effective amount of the immunogen of claim 5.

22. A method of inducing a protective immune response in a patient against a S. aureus infection comprising the step of administering to said patient an immunologically effective amount of the composition of claim 7.


SEQUENCE LISTING (6 sequences)

SEQ ID NO: 1 - protein, 114 aa, S. aureus
MAVNLYDYANQLEQALRESEEYKAIKEAFANVKANEESKKLFDEFRETQINFQQKQMQGEEIAEEDLQKAQEQAQAIEKDENISALMNAEQKMSQVFQEINQIIVKPLDEIYAD

SEQ ID NO: 2 - protein, 121 aa, Artificial Sequence
  His-tagged derivative of SEQ ID NO1
MGHHHHHHAVNLYDYANQLEQALRESEEYKAIKEAFANVKANEESKKLFDEFRETQINFQQKQMQGEEIAEEDLQKAQEQAQAIEKDENISALMNAEQKMSQVFQEINQIIVKPLDEIYAD

SEQ ID NO: 3 - DNA, 367 bp, Artificial Sequence
  cDNA sequence encoding SEQ ID NO2
atgggccatcatcatcatcatcacgcagtaaatttatatgattatgcaaatcaattagaacaagctttaagagaaagcgaagaatacaaagcaatcaaagaagcattcgctaatgtaaaagctaacgaagaatctaaaaagttattcgacgagttccgtgaaactcaaattaacttccaacaaaaacaaatgcaaggtgaagaaattgctgaagaagatttacaaaaagcgcaagaacaagcgcaagcaattgaaaaagatgaaaacatctctgcattaatgaatgctgaacaaaaaatgagtcaagtattccaagaaatcaaccaaattatcgttaaaccattagacgaaatttacgctgactaaa

SEQ ID NO: 4 - DNA, 21 bp, Artificial Sequence
  PCR primer
ttagtcagcgtaaatttcgtc

SEQ ID NO: 5 - DNA, 58 bp, Artificial Sequence
  PCR primer
atgggccatcatcatcatcatcacgcagtaaatttatatgattatgcaaatcaattag

SEQ ID NO: 6 - protein, 113 aa, S. aureus
MAVNIYDYANQLEQALRESEEYKAIKEAFANVKANEESKKLFDEFRETQINFQQKQMQEEIAEEDLQKAQEQAQAIEKDENISALMNAEQKMSQVFQEINQIIVKPLDEIYAD